(19) United States
(12) Patent Application Publication
(10) Pub. No.: US 2009/0246797 A1
(43) Pub. Date: Oct. 1, 2009

(54) MEDICAL DEVICE FOR THE ASSESSMENT OF INTERNAL ORGAN TISSUE AND TECHNIQUE FOR USING THE SAME

(75) Inventors: Shannon Campbell, Boulder, CO (US); Christina Rideout, Middletown, CT (US); Gilbert Hausmann, Felton, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/388,705

(22) Filed: Feb. 19, 2009

Related U.S. Application Data
(60) Provisional application No. 61/072,138, filed on Mar. 28, 2008.

(51) Int. Cl. G01N 33/53; C12M 1/34

(52) U.S. Cl. 435/7.1; 435/287.2

(57) ABSTRACT

A system for tissue ischemia detection is provided that may be used to assess markers of tissue ischemia. Such a system may include a sensor that may be used directly on internal tissue to assess ischemic condition. Sensors to be used in conjunction with the provided system may include optical, chemical or electrochemical sensors that may be directly applied or affixed to the tissue, held proximate to the tissue, or spread over the tissue in the form of a gel.

### CROSS-REFERENCE TO RELATED APPLICATION

This claims the benefit of U.S. Provisional Patent Application No. 61/072,138, filed Mar. 28, 2008, which is hereby incorporated by reference herein in its entirety.

### BACKGROUND

The present disclosure relates generally to medical devices and, more particularly, to sensors placed on a tissue surface for sensing the onset of an ischemia condition.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modern medicine.

During surgical procedures, the risk of infections and other complications is elevated. Internal infections after surgery can have consequences that include extended hospital stays and possible pharmaceutical intervention. For surgical procedures involving gastrointestinal tissue, such infection may be attributed to leakage of gastrointestinal contents after suturing or stapling intestinal tissues. Gastrointestinal contents have high concentrations of bacteria that, if leaked into the blood or abdomen, lead to inflammation and, potentially, infection.

A potential cause of a leak is tissue ischemia in the internal organs. Tissue ischemia may be an acute or a chronic condition caused by oxygen shortages in the tissue. Such oxygen shortages may lead to the degradation of tissue integrity, which may allow the contents of the gastrointestinal or other tissue to leak out into the surrounding space. For some patients, tissue ischemia may by the result of the underlying disease condition or injury that ultimately leads to a surgical procedure. Tissue ischemia may also be the result of the procedure itself. For example, tissue manipulation or a take down of blood supply during surgery may also contribute to tissue ischemia.

As a result, physicians may wish to have information about the clinical state of tissues that are difficult to access, such as gut tissue. For example, clinicians may wish to assess certain parameters of gut tissue to determine whether a patient is experiencing ischemia. Ischemia in the gut may disrupt the normal intestinal barrier function, resulting in gut-derived bacteria and endotoxins being able to move from the gut into other organs via the blood. This, in turn, may lead to toxemia or sepsis. Therefore, early detection of organ tissue damage may prevent the onset of organ failure or infection.

The current methods for detecting ischemic intestinal tissue include a general examination of the color and pulsation of the tissue with the naked eye or the use of laser Doppler to assess blood flow within the tissue. These approaches are problematic because they do not convey a finite measurement of local tissue health. For example, while laser Doppler may indicate that red blood cells are moving within the tissue, such measurements may not be useful for low-speed blood flow in the microvasculature. Further, although tissue integrity may be assessed by checking for leaks in the tissue, such techniques do not provide an accurate indication of the onset of ischemia in local organ tissue so that clinicians can prevent ensuing leaks and/or infection.

Accordingly, a reliable method for detecting the onset of organ tissue ischemia during surgery may improve the management of patients. This may minimize the complications associated with gastric and other surgery and may improve patient outcomes.

### SUMMARY

Certain aspects commensurate in scope with the originally claimed disclosure are set forth below. It should be understood that these aspects are presented merely to provide the reader with a brief summary of certain forms the disclosure might take and that these aspects are not intended to limit the scope of the disclosure. Indeed, the disclosure may encompass a variety of aspects that may not be set forth below.

There is provided a system for detecting tissue ischemia that includes a sensor capable of producing an output related to a tissue ischemia marker present in an internal tissue; and a processor capable of receiving and analyzing the output to determine a measurement of the tissue ischemia marker.

There is also provided a method of detecting tissue ischemia that includes generating a sensor output related to a tissue ischemia marker; and performing an operation on the sensor output to determine a presence, a change, and/or a level of the tissue ischemia marker.

There is also provided a method of detecting tissue ischemia that includes viewing a sensor comprising a hydrogel applied to an internal tissue, wherein a visual characteristic of the sensor corresponds to a present or level of a tissue ischemia marker; and determining if a presence or level of the tissue ischemia marker corresponds to an ischemic condition in the internal tissue based on the visual characteristic.

There is also provided a system for detecting tissue ischemia that includes a light source capable of directing light into an internal tissue; a light detector capable of detecting the light; and a processor operatively connected to the light detector and capable of analyzing the detected light to determine if the internal tissue is ischemic.

There is also provided a method for detecting tissue ischemia that includes directing a light onto a region of tissue; detecting the light reflected from the region of tissue; and analyzing the detected light to determine if a level of a tissue ischemia marker corresponds to an ischemic condition in the region of tissue.

### BRIEF DESCRIPTIONS OF THE DRAWINGS

Advantages of the disclosure may become apparent upon reading the following detailed description and upon reference to the drawings in which:

FIG. 1A illustrates a side perspective view of an optical patch sensor for detection of tissue ischemia in accordance with one embodiment;

FIG. 1B illustrates a side perspective view of an optical patch sensor with separate light detection and delivery fibers for detection of tissue ischemia in accordance with one embodiment;

FIG. 2 illustrates a schematic side view of a reflectance-type sensor for detection of tissue ischemia in accordance with one embodiment;

FIG. 3 illustrates an illustration of a light being directed onto a tissue to detect ischemia markers in accordance with one embodiment;

FIG. 4 illustrates a perspective view of a fiber optic stylet sensor for detection of tissue ischemia in accordance with one embodiment;

FIG. 5 illustrates a system for detection of tissue ischemia in accordance with one embodiment;

FIG. 6 illustrates block diagram of the system shown in FIG. 4 in accordance with one embodiment; and

FIG. 7 illustrates a view of a sensor that includes a hydrogel to indicate a tissue ischemia condition in accordance with one embodiment.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, for example compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Sensors and monitoring systems are provided herein that may detect ischemia by monitoring one or more markers for tissue ischemia, including the local levels of bilirubin, heat shock proteins (HSPs), carbon monoxide and/or iron in internal organ tissue. The levels of these markers in organ tissue may serve as an indicator or marker of tissue conditions including ischemia and hypoperfusion. Additionally, such sensors may be useful for predicting the onset of shock or organ failure. The disclosure provides a method of detecting the onset of ischemia in internal organs through direct measurement of biological factors present during these conditions. The monitoring systems detect the onset of the ischemic condition in local organ tissue before the ischemia becomes a systemic problem, which may lead to infection or other systemic conditions.

Moreover, in one embodiment, the systems discussed are for the assessment and/or detection of tissue ischemia during a surgical procedure. In such embodiments, the detection of ischemia can occur at any point, for example during pre-operative preparation through postoperative care. Generally, monitoring for ischemia may occur while a sensor is directly secured, pressed against, or proximate to the tissue of the organ to be monitored. A sensor may be attached by using an adhesive, clips, or other means. Multiple sensors may be deployed in a patient, depending on how many organs are to be monitored. In one embodiment, the measurement may also be taken by a stylet or wand, which would allow the analysis of more remote organ tissue.

Generally, it is envisioned that a sensor according to the present disclosure is appropriate for use in determining the presence or levels of tissue ischemia markers in organ tissue. Tissue ischemia markers may include any chemical compound, including proteins, peptides, enzymes, small chemical compounds, and/or metals, associated with detectable changes during ischemia. Such markers may include the enzyme heme oxygenase-1 (HO-1), sometimes referred to as hemoxygenase-1 or heat shock protein 32 (Hsp32), which may be induced by reactive oxygen metabolites and inflammatory cytokines. HO-1 catalyzes the breakdown of heme, releasing biliverdin. Biliverdin is then converted to bilirubin, carbon monoxide, and iron. Measuring local levels of the participants and products of such stress reactions may provide an early indication of internal organ ischemia, allowing for prompt treatment or other intervention. In certain embodiments, two or more tissue ischemia markers may be measured in concert.

Measured levels of tissue ischemia markers may be compared with normal or baseline measurements as an indication of tissue ischemia, infection, or inflammation. Alternatively, measurements of interest may include rates of change, changes relative to a predetermined index, or change relative to a patient-specific observed or monitored condition. For example, higher than normal levels of bilirubin in tissue may correlate with cell stress, tissue ischemia, and similar target conditions. Multiple measurements of tissue ischemia markers in different locations of the same or different tissue may also be used to provide an indication of healthy or unhealthy tissue. Concentrations of tissue ischemia markers may be measured as either a spot check or a continuous measurement. In addition, the measured levels of tissue ischemia markers may be displayed or otherwise communicated to medical staff, such as via an audible alarm and/or visual indicator. Treatment or intervention for ischemia, infection or inflammation may include local or systemic injections of drugs or other active agents, elevated concentration or pressure of oxygen or other gases, removal of the affected tissue, or other treatment based on clinical decisions.

Further, it is envisioned that the measurement of tissue ischemia markers may be made on any internal tissue or organ that may be susceptible to ischemia. Exemplary tissue may include gastrointestinal tissue, cardiac tissue, brain tissue, and/or kidney tissue. Although such internal tissue is generally unavailable for direct assessment, during surgical procedures while the body cavity is open, a clinician may apply a sensor to the exposed tissue in order to assess the presence of or changes in levels of tissue ischemia markers.

Measurement of the levels, concentration, or presence of tissue ischemia markers may be made using a variety of techniques. For example, such measurements may be performed using electrochemical sensors, chemical indicators, barometric measurements, optical detectors, fiber optic sensors, and other such devices. In one embodiment, the sensor may include an electromagnetic radiation emitter and detector that may be of any suitable type. For example, the emitter may be one or more light emitting diodes adapted to transmit one or more wavelengths of light, and the detector may be one or more photodetectors selected to receive light in the range or ranges emitted from the emitter. Alternatively, an emitter may also be a laser diode or a vertical cavity surface emitting laser (VCSEL). An emitter may include a broadband or “white light” source, in which case the detector could include any of a variety of elements for selecting specific wavelengths, for example reflective or refractive elements or interferometers. In one embodiment, of emitters and/or detectors may be coupled to a rigid or rigidified sensor via fiber optics.

Alternatively, in other embodiments, a sensor may sense light detected from the tissue at a different wavelength from the light emitted into the tissue. Such sensors may be adapted to sense fluorescence, phosphorescence, Raman scattering, Rayleigh scattering and multi-photon events or photoacoustic effects. It should be understood that, as used herein, the term “light” may refer to various forms of electromagnetic radiation, such as radio, microwave, millimeter wave, infrared, visible, ultraviolet, gamma ray or X-ray electromagnetic radiation, and may also include any wavelength within the radio, microwave, infrared, visible, ultraviolet, or X-ray spectra.

In one embodiment, nanotechnologies may be used in sensing the tissue ischemia markers of interest. Carbon nanotubes, buckeyballs, or other quantum restricted structures could be functionalized with one or more specific ligands for the purpose of specifically binding the marker of interest, such as bilirubin, carbon monoxide or iron. Binding agents for bilirubin, such as bilitranslocase, or polymers that react with carbon monoxide are examples of the functional moieties that could be used in such an embodiment. Other embodiments may include nano-structures coated with a film of materials that are reactive with the measured marker and produce a secondary effect on the properties (electrical and/or optical) of the above-mentioned nano-structure.

In one embodiment, the sensors may also include an enzyme-based detection system. For example, one such enzyme-based detection system is an enzyme linked immunosorbent assay (ELISA). For example, such an assay may be appropriate when assessing proteins. Thus, in one embodiment, the indicator element may include a primary antibody specific for the tissue protein of interest, such as an antibody specific for HO-1, and a labeled secondary binding ligand or antibody, or a secondary binding ligand or antibody in conjunction with a labeled tertiary antibody or third binding ligand. The label may be an enzyme that will exhibit color development upon incubating with an appropriate chromogenic substrate. Suitable enzymes include urease, glucose oxidase, alkaline phosphatase or hydrogen peroxidase.

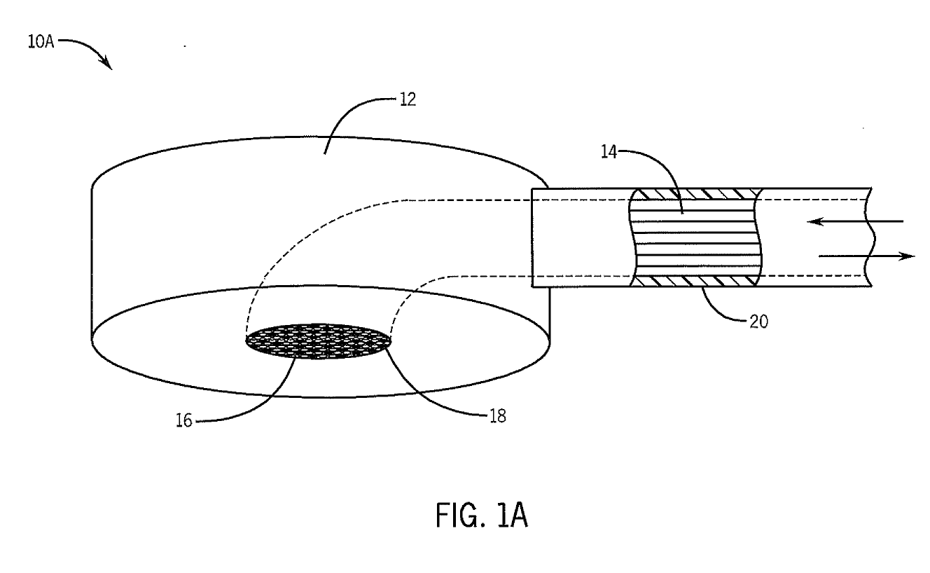

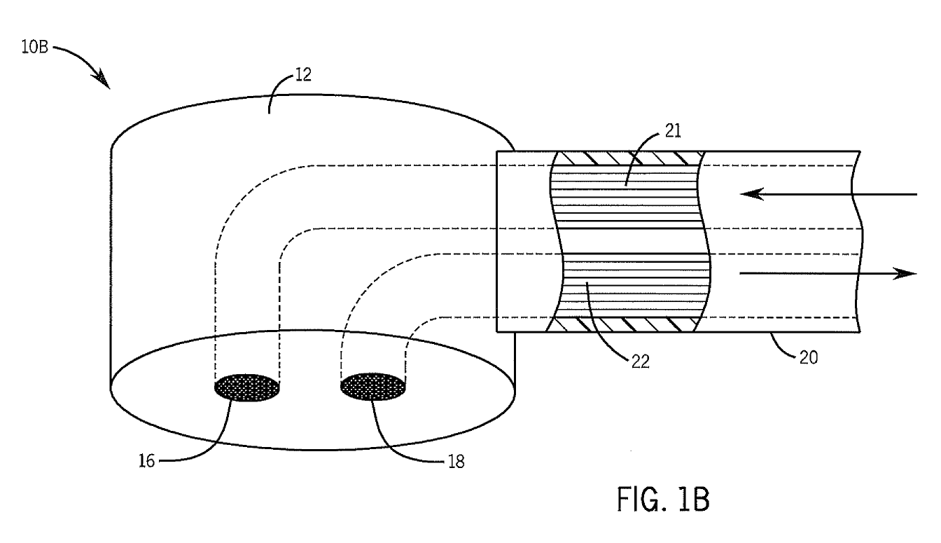

With the foregoing discussion of sensors and sensor technologies in mind, reference is now made to FIG. 1A. In FIG. 1A, an embodiment is depicted including a sensor 10A that is shown in the form of an optical patch sensor. Sensor 10A includes sensor body 12 and bundle of fibers 14, which include light delivery fibers 16 and light collection fibers 18. The bundle of fibers 14 carry the light emitted and detected by sensor 10A to a monitor (not pictured) via fiber optic lead 20. In an embodiment shown in FIG. 1B, sensor 10B includes light delivery fibers 16 bundled separately from light collection fibers 18. The fibers are correspondingly separated as light delivery fibers 21 or light collection fibers 22 within the fiber optic lead 20.

In one embodiment, the sensor 10A or 10B may operate by directing white light into the tissue and measuring the intensity of the specific wavelengths that are returned. The absorption of photons by bilirubin occurs at a wavelength of between about 400 nm to about 500 nm with peak absorption at about 460 nm, and light measured in this range may therefore be utilized in measuring bilirubin in the illuminated tissue. The sensor may also direct a reference wavelength into the tissue for the purpose of subtracting out absorbance peaks from other compounds. For example, a reference wavelength may be around 585 nm because bilirubin does not absorb at 585 nm. The choice of 585 nm may be useful for subtracting out the absorption by blood. Further, additional reference wavelengths may also be used. Once the absorption due to bilirubin is calculated, the average bilirubin concentration in the tissue may be calculated according to techniques provided in U.S. Pat. No. 5,353,790, which is herein incorporated by reference in its entirety for all purposes.

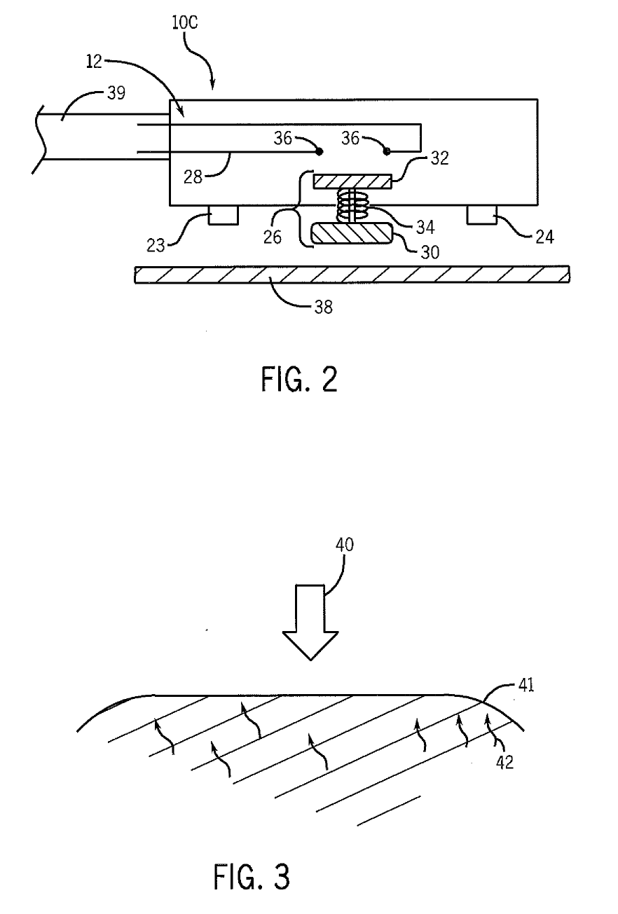

FIG. 2 is an illustration of a reflectance-type sensor 10C according to certain embodiments. The sensor 10C includes light emitter 23 and light detector 24. The sensor 10C may also include a tissue contact indicator, such as a plunger assembly 26 that may be capable of closing a circuit 28 upon proper application of the sensor 10C to the tissue site. Such a sensor 10C may be advantageous for providing an indication to a clinician that the sensor is properly placed on the tissue during the surgical procedure. Because a clinician may manipulate the tissue during surgery, which may result in movement of the sensor 10C on the tissue, a sensor contact indicator may provide information to the clinician about changes in sensor placement or sufficiency of sensor contact. The exemplary plunger assembly 26 includes a tissue contact element 30, a biasing member 34, and a switch element 32. Generally, the switch element 32 may be formed from any suitable conductive material, such as a metal. The tissue contact element 30 may be formed from any suitable biocompatible material, such as a metal or thermoplastic polymer, that may be sufficiently resilient to transmit pressure from the tissue to the biasing member 34. The plunger assembly 26 may be biased by the biasing member 34, such as a spring, such that the switch element 32 will not close the circuit 28 without sufficient pressure being applied to the tissue contact element 30. The spring may be sized such that when the sensor 10 is properly applied against a monitoring site, the plunger assembly 26 may move, and the switch element 32 will close the circuit 28 across the contacts 36. The monitoring site may be the tissue 38 of the organ to be evaluated. In such an embodiment, the closed circuit may correspond to the “sensor on” state. The signals indicating levels of bilirubin or other tissue ischemia marker being monitored are acquired by light emitter 23 and light detector 24 and are transmitted to a monitor (not shown) via lead 39.

In one embodiment, a clinician may monitor an internal tissue for the presence of tissue ischemia markers by taking advantage of the natural chemilumiscent properties of bilirubin. Bilirubin in an alkaline solution exhibits chemiluminescence under aerobic conditions. Accordingly, an exposed internal organ with tissue ischemia may exhibit chemilumiscent properties under certain conditions. As illustrated in FIG. 3, a clinician may shine light, illustrated by arrow 40, of the appropriate excitation wavelength, for example light of about 350 nm, onto an internal tissue 41 during a surgical procedure. An increase in chemilumiscent emissions, illustrated by arrows 42, around a peak of about 670 nm may indicate an increase in bilirubin in the tissue, and thus may be an indicator of tissue ischemia. In some embodiments, emission and detection of the relevant light wavelengths may be accomplished using a suitable sensor, such as sensors 10A, 10B, 10C, and/or sensor 10D as described herein. In other embodiments, light emission may be performed using a hand-held emitter away from the tissue such that an expanse of the tissue is illuminated with the desired wavelength of light. In such embodiments, detection of the chemiluminescent emissions may be by eyesight or by a suitable electrical or optical detector. Chemilumiscence may be increased by factors such as pH or by the presence of aldehydes. In one embodiment, a clinician may apply a biocompatible alkaline liquid or gel to the tissue prior to exposure to excitation light. In one embodiment, a clinician may apply a biocompatible functionalized aldehyde-containing compound to the tissue, for example aldehyde-functionalized hyaluronic acid prior to exposure to excitation light.

A sensor, for example sensor 10A, 10B, and/or 10C, may be affixed to internal tissue during a surgical procedure and/or monitoring period with a mucoadhesive that may include a variety of mucoadhesive compositions. Suitable mucoadhesives include, but are not limited to hydroxypropyl cellulose, hydroxypropyl methylcellulose, hydroxyethylcellulose, ethylcellulose, carboxymethylcellulose, dextran, guar gum, polyvinyl pyrrolidone, pectins, starches, gelatin, casein, acrylic acid polymers, polymers of acrylic acid esters, vinyl polymers, vinyl copolymers, polymers of vinyl alcohols, alkoxy polymers, polyethylene oxide polymers, polyethers, or any combination of the above.

In specific embodiments, the mucoadhesive may include a biocompatible polymer, for example polyacrylic acid, that is cross-linked with an acceptable agent to create an insoluble gel. In such an embodiment, the insoluble gel may remain adhered to the mucosal tissue for relatively long periods of time. For example, cross-linked polyacrylic acid polymers, such as Noveon® and Carbomer®, may be appropriate for use for three to five days or longer. Noveon® and Carbomer®-based polymers are weak acids and contain many negatively-charged carboxyl-groups. The multiple negative charges on these polymers promote hydrogen-bonding between the polymers and the negatively charged mucin, a glycoprotein that mediates attachment of mucus to the epithelial lining.

A mucoadhesive polymer may also include acrylic acid polymers (e.g. Carbopol® 940, also known as Carbomer® 940, Carbopol® 934P and Carbopol® 980, products of BF Goodrich®), methyl vinyl/maleic acid copolymers (e.g. Gantrez® S-97, a product of International Specialty Products), and/or polyvinyl pyrrolidone also known as povidone (e.g. Plasdone® K-90, a product of International Specialty Products). These polymers impart relatively high viscosity at relatively low concentrations. They may therefore be incorporated onto a sensor in amounts ranging from about 0.01% to about 10% by weight relative to the total composition. These viscosity modifying agents further act to improve the film adhesion of the composition to mucous membranes. For example, Carbopol® 980, in certain embodiments, may be 2-3% by weight of the total composition. The mucoadhesive may be formulated as either a liquid or as a gel. If a liquid formulation is desired, a relatively low concentration (e.g. 0.1-1%) of the mucoadhesive/viscosity modifying agent may be used. If a gel formulation is desired, a higher concentration (e.g. 1.5-4%) of the suitable viscosity modifying/mucoadhesive agent may be incorporated into the polymethacrylate/solvent vehicle for gel formation.

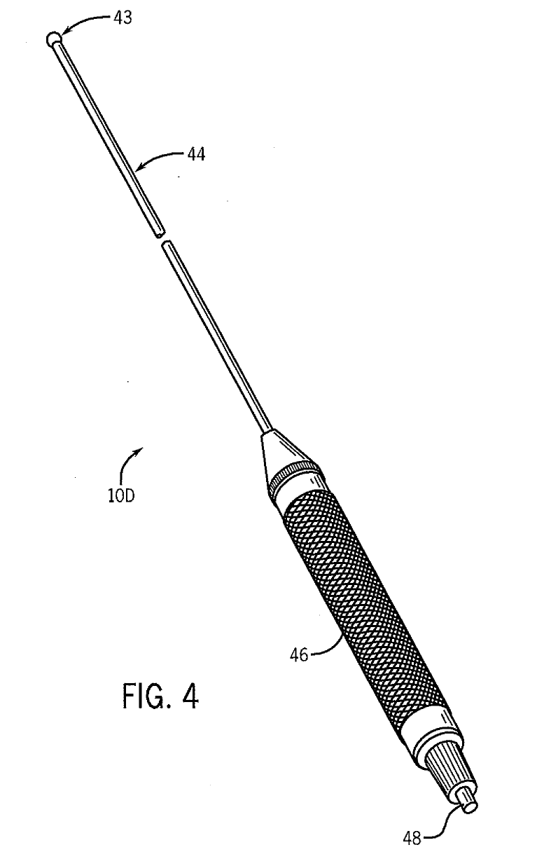

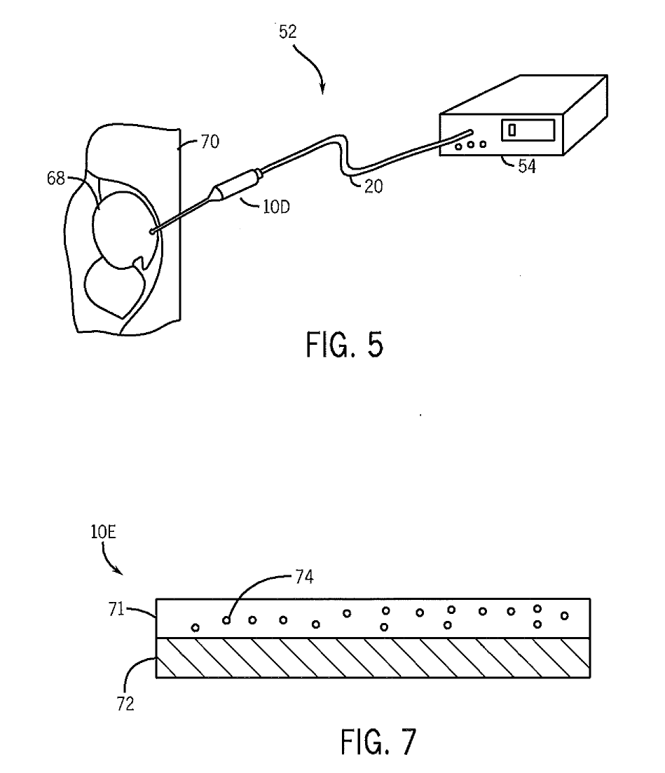

An embodiment is shown in FIG. 4 in which a stylet sensor 10D may be used to detect the presence and/or levels of a tissue ischemia marker such as bilirubin. Sensor tip 43 is designed to be applied to organ tissue and may include fiber optics to emit and detect light waves. Light waves are transmitted from sensor tip 43 via fiber optic wand 44 to sensor handle 46. Sensor handle 46 and wand 44 may be encased in any suitable durable material, including plastics, composites, metal, or combinations of these materials. A fiber optic lead 48 transmits the light signal to a monitor (not shown). Stylet sensor 10D may be advantageous for examining tissue located in portions of the body cavity remote to a clinician. For example, a surgeon may detect an ischemic condition in heart tissue by inserting the stylet sensor 10D through a small incision made near a patient's kidney and manipulating the sensor tip 43 until proximate to the heart tissue. The use of sensor 10D in conjunction with medical system 52 is depicted in FIG. 5. In the depicted example, the stylet sensor 10D is pressed against gut tissue 68 of patient 70 during a surgical procedure. An ischemia condition may be indicated by a visual and/or audio alarm on monitor 54, discussed in more detail below.

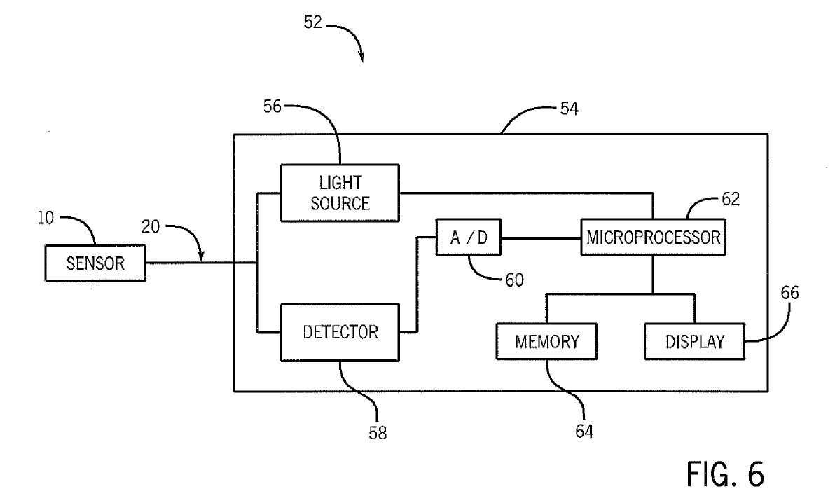

FIG. 6 depicts a block diagram of the medical system 52 shown in FIG. 5. In this embodiment, the light signal from the sensor 10, corresponding to a tissue ischemia marker, is transmitted by lead 20 to monitor 54. In addition, in this embodiment, lead 20 also communicates emitted light between monitor 54 and sensor 10. For example, as depicted, the monitor 54 may include light source 56 and light detector 58. In other embodiments (not shown), the sensor 10 may include a light source, such as an LED, as well as a detector. In such embodiments, an electrical or optical signal representative of detected light may be transmitted by lead 20. Analog to digital converter 60 may also be included to convert the acquired signal to a format to be interpreted and analyzed by microprocessor 62. As will be appreciated by one of ordinary skill in the art, the measurement data may be stored in a memory 64 and/or may also displayed on a display 66 as a measured value or as an alarm when appropriate.

In one embodiment, the monitor 54 may be configured to receive signals from the sensor 10 related to the levels of a tissue ischemia marker in a patient's tissue. Based on the value of the received signals, corresponding to detected light waves, the microprocessor 62 may detect an ischemic condition using various algorithms. For example, in one embodiment, the memory 64 may contain comparison charts or tables accessed by the processor 62 for comparing measured bilirubin levels or bilirubin level changes with clinically-derived values that may correlate with specific ischemic states. In certain embodiments, the measurement of the tissue ischemia may be displayed as a numerical value. In other embodiments, an algorithm may use as an input bilirubin data to output a qualitative indicator that corresponds to a patient clinical condition.

For example, a threshold bilirubin level value, based on reference data, may be used for comparison to measured bilirubin levels. In a qualitative display embodiment, as long as the measured value remains well below the threshold value, the monitor 54 may illuminate a corresponding green light, indicating a “healthy” tissue status. If the measurement value is within a predetermined range, e.g. within 5% of the threshold, then a yellow light may be illuminated, signaling a “warning” status. The “warning” range may correlate to an acceptable standard variance, as determined by statistical analysis of bilirubin level data. Finally, if the measurement exceeds the threshold bilirubin level, a red light may be displayed, indicating an “alarm” status and a clinical condition of gut tissue ischemia.

The analysis of the bilirubin level measurement data may include a threshold comparison to the raw data. Alternatively, the analysis may include a comparison of the threshold to a running average or mean of the measurement so that measurement errors are less likely to result in false alarms. The aforementioned analysis may correspond to an embodiment where predetermined reference data is used to establish a threshold bilirubin level value for comparison. In another embodiment, the measured bilirubin level data may be monitored for changes in value indicating a change in condition. In such an embodiment, a threshold may be established for an alarm by a change that exceeds a certain percentage difference, as compared to a prior reading. Again, the bilirubin level change data may use raw values, comparing successive readings, or the data may constitute a comparison of the running average to an average of prior data, which may reduce false alarms.

In one specific embodiment, the disclosed method of detecting ischemia in the gut may be used throughout a surgical procedure. In such an embodiment, a plurality of baseline measurements of the mean tissue ischemia marker level may be recorded) for example over a five or ten minute period prior to the surgery. These baseline measurements may then be used to compute a baseline value of mean tissue ischemia marker level as well as the standard variance. After the data are collected for determining the baseline value and the variance, the levels of the marker in the tissue may be periodically measured over the course of the surgery. The periodically measured levels may be compared against the stored baseline values. If the measured level varies from the baseline and/or previously determined standard variance, an alarm may be triggered to alert the surgeon to possible ischemia in the gut, as discussed above.

While certain of the above embodiments describe types of sensors that detect optical changes, other embodiments are also possible that may detect a combination of chemical and optical changes. In one embodiment, the sensor 10 may take the form of a gel or other spreadable compound that may be applied to a relatively widespread area of tissue. Such an embodiment may provide the advantage of allowing a clinician to assess tissue health over all or part of an organ. Illustrated in FIG. 7 is an embodiment in which a sensor 10E includes a hydrogel 71 that may be applied to organ tissue 72. The hydrogel 71 may be any suitable hydrogel into which is dispersed an indicator element 74 adapted to provide an output in the presence of a tissue ischemia marker. Alternatively, the hydrogel 71 may be functionalized with the indicator element 74, such that the indicator element 74 is directly attached to or interpenetrated within the hydrogel 71.

In an embodiment, the indicator elements 74 may include a chromogenic indicator such as a diazonium salt. Bilirubin, in the presence of certain diazonium compounds, cleaves at its central methylene group to form two molecules of pigment known as azobilirubin. These molecules appear blue to purple at acid and basic pH and red at neutral pH. The hydrogel may be modified as desired to provide an appropriate acidity by modifying the hydrogel monomers and/or the monomer solution. One such diazonium salt that may be employed in such an embodiment is 5-dichlorophenol diazonium tetrafluoroborate. In order to improve stability of the chromogenic indicator, suitable additives may also be present in the hydrogel 71. In an embodiment, the additives may include aryl-sulfonic acids or meta-phosphoric acid. The clinician may view a chromogenic change in all or part of the applied sensor 10E to determine if the level of the tissue ischemia marker has increased. In an embodiment, a clinician may view a color change to a blue/purple color in the presence of bilirubin. In one embodiment, one or more optical sensors may be placed within the hydrogel 71 to provide an electrical output related to the color change.

In such an embodiment, the hydrogel 71 may include carboxymethyl cellulose, polyethylene glycol polymers, silicone gels, or other biocompatible gel-forming materials. The hydrogel 71 may also include a polymer of N-isopropylacrylamide (N-IPAM). In one embodiment, the hydrogel 71 includes hyaluronic acid, which is a naturally occurring linear polysaccharide composed of alternating disaccharide units of N-acetyl-D-glucosamine and D-glucuronidic acid. Hyaluronic acid is widely distributed in animal tissues, present in high concentrations in synovial fluid and the vitreous body of the eye, and in connective tissues of rooster comb, umbilical cord, and dermis. The term hyaluronic acid may include any hyaluronate salts, including, sodium hyaluronate, potassium hyaluronate, magnesium hyaluronate, and calcium hyaluronate.

The hydrogel 71 may include, for example, gels of hyaluronan (hyaluronic acid) cross-linked with vinyl sulfone or cross-linked mixtures of hyaluronan with other polymers or low molecular weight-substances. The hydrogel 71 may include hydrolysable groups such as include polymers and oligomers of glycolide, lactide, epsilon-caprolactone, other hydroxy acids, and other biologically degradable polymers that yield materials that are non-toxic or present as normal metabolites in the body. Examples of such polymers include poly(alpha-hydroxy acids), poly(glycolic acid), poly(DL-lactic acid) and poly(L-lactic acid).

While the disclosure may be susceptible to various modifications and alternative forms, specific embodiments have been shown by way of example in the drawings and have been described in detail herein. However, it should be understood that the disclosure is not intended to be limited to the particular forms disclosed. Indeed, the present techniques may not only be applied to measurements of tissue ischemia markers, but these techniques may also be utilized for the measurement and/or analysis of other tissue constituents. Rather, the disclosure is to cover all modifications, equivalents, and alternatives falling within the spirit and scope of the disclosure as defined by the following appended claims. It will be appreciated by those working in the art that sensors fabricated using the presently disclosed and claimed techniques may be used in a wide variety of contexts. That is, while the disclosure has primarily been described in conjunction with the measurement of tissue ischemia markers in internal tissue, the sensors fabricated using the present method may be used to evaluate any number of tissue markers in a variety of applications.

What is claimed is:

1. A system for detecting tissue ischemia comprising:
a sensor capable of producing an output related to a tissue ischemia marker present in an internal tissue; and
a processor capable of receiving and analyzing the output to determine a measurement of the tissue ischemia marker.

2. The system recited in claim 1, wherein the output comprises an electrical output, an optical output and/or a colormetric output.

3. The system recited in claim 1, wherein the tissue ischemia marker comprises bilirubin, heat shock protein 32, and/or carbon monoxide.

4. The system recited in claim 1, wherein the sensor comprises an antibody with affinity for the tissue ischemia marker.

5. The system recited in claim 1, wherein the sensor comprises a hydrogel.

6. The system recited in claim 1, wherein the sensor comprises one or more light emitters and one or more light detectors.

7. The system recited in claim 1, wherein the processor determines if the tissue is ischemic based on the measurement of the tissue ischemia marker.

8. A method of detecting tissue ischemia comprising:
generating a sensor output related to a tissue ischemia marker; and
performing an operation on the sensor output to determine a presence, a change, and/or a level of the tissue ischemia marker.

9. The method recited in claim 8, comprising determining if the tissue is ischemic based on the presence, change, and/or level of the tissue ischemia marker.

10. The method recited in claim 9, comprising triggering an alarm if the level of the tissue ischemia marker corresponds to the ischemic condition.

11. The method recited in claim 8, wherein the ouput comprises an optical output.

12. A method of detecting tissue ischemia comprising:
viewing a sensor comprising a hydrogel applied to an internal tissue, wherein a visual characteristic of the sensor corresponds to a present or level of a tissue ischemia marker; and
determining if the presence or level of the tissue ischemia marker corresponds to an ischemic condition in the internal tissue based on the visual characteristic.

13. The method recited in claim 12, comprising shining a fluorescent light on the hydrogel, and wherein determining the presence or level of the tissue ischemia marker comprises detecting a change in fluorescence.

14. The method recited in claim 13, wherein detecting the change in fluorescence comprises detecting an increase in chemiluminescence of bilirubin.

15. A system for detecting tissue ischemia comprising:
a light source capable of directing light into an internal tissue;
a light detector capable of detecting the light; and
a processor operatively connected to the light detector and capable of analyzing the detected light to determine if the internal tissue is ischemic.

16. The system of claim 15, wherein the analysis comprises detecting a level of bilirubin and/or carbon monoxide.

17. The system of claim 15, wherein the analysis comprises comparing the detected light to a predetermined threshold value.

18. The system of claim 15, wherein the light comprises a wavelength between about 400 nm and about 500 nm.

19. The system of claim 19, wherein the light analyzed comprises a peak wavelength of about 460 nm.

20. The system of claim 15, wherein the analysis comprises monitoring an ischemia marker to determine a quantitative level of the ischemia marker.

18. The system of claim 15, wherein the analysis comprises monitoring an ischemia marker over time to determine a rate of change of the ischemia marker.

22. The system of claim 15, wherein the analysis comprises monitoring an ischemia marker to determine a presence of the ischemia marker.

23. A method for detecting tissue ischemia, the method comprising:
directing a light onto a region of tissue;
detecting the light reflected from the region of tissue; and
analyzing the detected light to determine if a level of a tissue ischemia marker corresponds to an ischemic condition in the region of tissue.

24. The method of claim 23, comprising comparing the tissue ischemia marker level to a predetermined threshold value.

25. The method of claim 23, comprising monitoring a rate of change in the tissue ischemia marker level.
